(19) United States
(12) Patent Application Publication
(10) Pub. No.: US 2008/0183291 A1
(43) Pub. Date: Jul. 31, 2008

(54) Resurfacing the tibial plateau

(75) Inventors: Arnold Douglas Scheller, Milton, MA (US); Peter Krijger, Rockaway, NJ (US); Michael N. Campbell, Elmwood, NJ (US); Jerry D'Alessio, Belleville, NJ (US); Eric Jones, Limerick (IE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/699,321

(22) Filed: Jan. 29, 2007

(51) Int. Cl. A61F 2/38

(52) U.S. Cl. 623/14.12; 623/908; 623/20.32

(57) ABSTRACT

A meniscus implant having a compressible bearing element with an articulation surface. The implant also includes a bone-securing element extending downwardly from the bearing element and configured to be engaged within a channel created within a tibial plateau.

### BACKGROUND OF THE INVENTION

The present invention pertains to prosthetic devices. More particularly, the invention pertains to knee joint prosthesis, which may be surgically implanted between the femoral condyle and tibial plateau of the knee joint.

A meniscal cartilage provides the mobile weight bearing surfaces of the knee joint. Damage to these surfaces is generally due to genetic predisposition, trauma, and/or aging. The result is usually the development of chondromalacia, thinning and softening of the articular cartilage, and degenerative tearing of the meniscal cartilage. Various methods of treatment are available to treat these disease processes. Each option usually has specific indications and is accompanied by a list of benefits and efficiencies that may be compared to other options.

The healthy knee joint has a balance of joint cartilage across the four surfaces of this bi-compartmental joint (medical femoral condyle, medial tibial plateau, lateral femoral condyle and lateral tibial plateau). In patients with osteoarthritis, knee degenerative process typically leads to an asymmetric wear pattern that leaves one compartment with symmetrically less articular cartilage covering the distal portions (or weight bearing area) of the tibia and the femur than the other compartment. Most commonly, the medial compartment of the knee joint is affected more often than the lateral compartment.

As the disease progresses, large amounts of articular cartilage are worn away. Due to the asymmetrical nature of the erosion, the alignment of the mechanical axis of rotation of the femur relative to the tibia becomes tilted down towards the compartment which is suffering the majority of the erosion. This results in VARUS (bow-leg) deformity in the case of a medial compartment disease predominates, or a VALGUS (knock-kneed) deformity in a case of lateral compartment disease predominance. Factors such as excessive body weight, previously traumatic injury, knee instability, the absence of meniscus and genetic predisposition, all affect the rate of the disease.

It is important to understand that the disease manifests itself as periodic continuous pain that can be quite uncomfortable for the patient. The cause of this pain is subject to many opinions, but it is apparent that, as the joint compartment collapses, the collateral ligament on the side of the predominant diseased area becomes increasingly slack (like one side of a pair of loose suspenders), and the tibial and femoral axis move, for example, from a VALGUS to VARUS condition. This increases the stress of the opposing collateral ligament (and cruciate ligaments as well) and shifts the load bearing function of this bi-compartmental joint increasingly towards the disease side. This increasing joint laxity is suspected as causing some of the pain one feels. In addition, as the bearing loads are shifted, the body responds to the increased loading of the diseased compartment with increased production of bony-surfaced areas in an attempt to reduce the ever-increasing area unit loading. All of the shifting of the knee component geometry causes a misalignment of the mechanical axis of the joint. The misalignment causes an increase in the rate of degenerative change to the diseased joint surfaces causing an ever-increasing amount of cartilage debris to build up in the joint, further causing joint inflammation and subsequent pain.

Currently there is a void in options to treat the relatively young patient with moderate to severe chondromalacia involving mainly one compartment of the knee. Current treatments include cortisone injections, hyaluronic acid (HA) injections and arthroscopic debridement. Repeated cortisone injections actually weaken articular cartilage after a long period of time. HA has shown promising results but is only a short-term solution for pain. Arthroscopic debridement alone frequently does not provide long-lasting relief of symptoms. Unfortunately, the lack of long-term success of these treatments leads to more invasive treatment methods. Osteochondral allografts and micro fracture techniques are indicated for small cartilage defects that are typically the result of trauma. These procedures are not suitable for addressing large areas of degeneration. In addition, osteochondral allografts can only be used to address defects on the femoral condyle. Tibial degeneration can not be addressed with this technique.

The only true solution is to rebuild the defective joint by (filling) the joint space with more articular bearing material through complete resurfacing of the existing femoral condyle and tibial plateau. By replacing the original cartilage to its pre-disease depth, the joint mechanical axis alignment is restored to its original condition. Unfortunately these natural articular materials and surgical technology required to accomplish replacement tasks do not yet exist.

Therefore, what is needed is a uni-compartmental interpositional spacer, which by effectively replacing worn articular material, restores normal joint alignment and provides an anatomical correct bearing surface for the femoral condyle to articulate against.

### SUMMARY OF THE INVENTION

The present invention is directed toward the method of performing surgery and various implants that may be used during knee reconstruction or surgery. In one aspect of the present invention, the method of performing surgery may include forming a groove in a tibial plateau. Next, an implant having a bone-securing element and an articulation element is provided and placed within the groove. Specifically, the bone securing element of the implant is positioned within the groove such that the articulation element is disposed adjacent a tibial plateau. In one aspect, the groove extends from the anterior of the tibia to the posterior. While forming a groove, at least some damaged cartilage may be removed from the tibial plateau.

The groove may include a first side extending from the medial to lateral side and a second width extending in the same direction. And the first width may be larger than the second width. The bone-securing element of the implant may include a corresponding geometric shape that is similarly shaped to the geometric shape of the groove.

In yet another aspect of the present invention, the implant may include an intermediate portion that is attached to both the bone-securing element and the articulation element such that the intermediate portion connects the articulation element to the bone-securing element.

Another aspect of the present invention, a meniscus implant may include a compressible bearing element having an articulation surface and a bone-securing element extending downwardly from the bearing element and configured to be engaged within a channel created within a tibial plateau.

At least a portion of the compressible bearing element may be embedded within a portion of the bone-securing element. And the bone-securing element may have a porosity that promotes bone ingrowth. The bone-securing element may include a first side wall and a second side wall. Each of the side walls may include a transitional portion that transitions the first side and second side walls from a separation that is equal to a first distance to a separation that is equal to a second distance.

### BRIEF DESCRIPTION OF THE DRAWINGS

FIG. 1 is a side view of one embodiment of the present invention;

FIG. 2 is a top perspective view of the embodiment of FIG. 1;

FIG. 3 is a bottom perspective view of the embodiment of FIG. 1;

FIG. 4 illustrates a step according to one method of the present invention;

FIG. 5 illustrates an additional step according to the method also illustrated in FIG. 4;

FIG. 6 illustrates the method as discussed with regards to FIGS. 4 and 5 however done on an opposite side of the tibial plateau; and

FIGS. 7 and 8 illustrate a perspective and side view, respectively of yet another alternate embodiment of the present invention.

### DETAILED DESCRIPTION

The prosthesis meniscal devices of the subject invention are uni-compartmental devices suitable for minimally invasive, surgical implantation. By the term “meniscal” it is meant that the devices are positioned within a compartment in which a portion of the natural meniscus is ordinarily located. The natural meniscus may be maintained in position or may be wholly or partially removed, depending upon its condition. Under ordinary circumstances, pieces of a natural meniscus, which have been torn away, are removed, and damaged areas may be trimmed as necessary. In somewhat rare instances, the entire portion of the meniscus residing in meniscal cavity may be removed. Thus, the term “meniscal device” is descriptive of the location of the device rather than implying that it is a replacement for or has the shape of the natural meniscus. In most cases, the meniscal device of the present invention does not have the same shape as the natural meniscus and will not entirely replace the meniscus.

By the term “uni-compartmental” it is meant that each device is suitable for implantation into but one department defined by the space between the femoral condyle and it's associated tibial plateau. In other words, the device is not a “bi-compartmental” device which, in one rigid device, could be inserted into both of the two femoral condyle/tibial plateau compartments, unless it is specifically called out that the device is bi-compartmental. In many, if not most cases, a device will be inserted into one compartment only, generally the medial compartment, as meniscus and associated articular surfaces in these components (left knee medial and right knee medial compartments) are most subject to wear and damage. However, it is possible to insert two separate devices into the medial and lateral compartments of the same knee, or to use two such devices that are mechanically but not rigidly bi-compartmental.

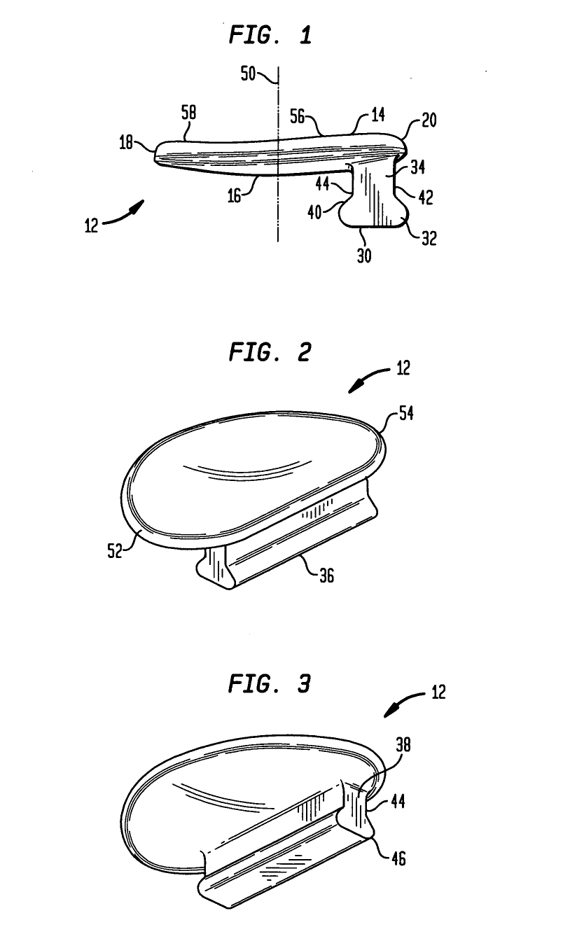

With reference to FIGS. 1-3, a first embodiment of an implantable prosthesis is illustrated in the form of a meniscal implant 12. The meniscal implant 12 includes a femoral facing surface 14 and an oppositely-facing tibial surface 16. These two surfaces generally are convex or concave. For instance, as shown in FIGS. 1-3, the femoral facing surface 14 is concaved so as to provide an articulation surface for a femoral condyle as will be described below. In contrast, the tibial facing surface 16 has a generally convex shape so as to conform to the distal end of a tibial plateau. The femoral facing surface 14 and tibial facing surface 16 are in communication via an edge 18 extending between the two surfaces 20 of the meniscal implant. The femoral facing surface 14, tibial facing surface 16 and edge 18 comprise the soft-flexible articulation portion 20 of the meniscal implant. The “articulation portion” refers to the part of the implant that either replaces or supplements the normal cartilage found on a tibial plateau.

The articulation portion 20 is generally flexible and is preferably made from a polymer such as polyurethane, Delrin, Ultem, PVA, PEEK, and/or polyethylene. The articulation portion 20 thus uses a flexible, low profile layer of smooth material to create a smooth articulating surface as defined by the femoral facing surface 14. The femoral facing surface 14 is designed to slide against mating cartilage, bone, and/or additional implants. Preferably the new artificial joining surface created by the femoral facing surface 14 supports any type of motion that naturally occurs within the older joint surface prior to the damage that was incurred.

The meniscus implant 12 further includes a securing element such as a keel 30. Keel 30 is preferably comprised of a three dimensional titanium mesh having a predetermined porosity. The keel includes a bone anchoring portion 32 and an intermediate portion 34. The keel 30 may be constructed using various methods known to those in the art. And in certain preferred environments, the keel 30 may be formed using methods as described and commonly assigned U.S. patent application Ser. Nos. 11/448,954 entitled “Flexible Joint Implant”; 10/704,270 entitled “Laser-Produced Porous Surface”; 11/027,421 entitled, “Gradiant Porous Implant”; 11/295,008 entitled “Laser-Produced Porous Surface”, the disclosures of which are hereby incorporated by reference herein.

As discussed in U.S. patent application Ser. No. 10/704,270, the keel 30 may be constructed using a selective laser melting or sintering process, which hereby grows the structure in a layer by layer process. In an alternate process, the keel 30 may be built using a method described in U.S. patent application Ser. No. 10/704,270, wherein the intermediate portion 34 acts as a base or substrate on which the bone-anchoring portion 32 is built, also in a layer by layer fashion. Additional techniques for constructing metalized structures i.e., keel 30, may also be constructed employing methods as disclosed in commonly assigned in U.S. patent application Ser. No. 10/071,667 entitled “Porous Metallic Scaffold for Tissue”, the disclosure of which is hereby incorporated by reference herein, as well as additional methods as known to those in the art such as that disclosed in Patent Cooperation Treaty Application 2005/023118 entitled “Porous Metal Articles Formed Using an Extractable Particulate” filed on Jul. 22, 2004 the disclosure of which is hereby incorporated by reference herein.

The keel 30 generally has a height from a first end 36 of the keel to a second end 38 of the keel of approximately 4 mm to 20 mm The bone-engaging portion 32 of the keel 30 preferably has a height of between 2 mm and 18 mm.

As shown in FIG. 1, the keel 30 includes two opposing side walls 40 and 42. The opposing side walls 40 and 42 are generally symmetrical although this is not required. Each side wall 40, 42 preferably defines a narrow portion 44 and a wide portion 46. The narrow portion 44 may be substantially placed within the intermediate portion 34 of the keel 30 while the wide portion 46 may be substantially placed within the bone-anchoring portion 32 of the keel. As shown in FIG. 1, the side walls 40 and 42 preferably may include a curved transition wall 44 that allows a smooth transition between the narrow portion 44 of the keel to the wide portion 46. Although the transition wall is illustrated as being curved, the wall may be straight, slanted or have different configurations, as well as including steps and the like. Similarly, the keel 30 may simply include two straight side walls thereby giving the keel a constant width as opposed to the varying width shown in the figures.

With reference to FIG. 1, a longitudinal plane 50 slices the meniscus implant 12 in half. The longitudinal plane 50 extends from the anterior 52 to the posterior 54 of the implant. Thus, the longitudinal plane 50 dissects the meniscus implant 12 into an inside segment 56 and an outside segment 58. The reference to an “inside segment” or “outside segment” has no geometry meaning as to the superior articular surface onto which the meniscus implant 12 may be positioned, be it on the medial or lateral side of the tibial plateau. But rather the inside segment refers to the fact that the meniscus implant 12 will be positioned with the inside segment 56 being closer positioned to a line passing through a mechanical axis of the tibia in a lengthwise direction while the outside segment 58 is farther away from the mechanical axis.

In FIGS. 1-3, the keel 30 is positioned offset from a longitudinal plane 50 and adjacent to the edge 18 of the articulation portion 20 of the implant that is within the inside segment 56 of the implant. The keel 30, however, may be positioned at any position or in any orientation relative to the articulation portion 20 including centrally. Therefore, as shown in the figures, the keel extends from the anterior 52 to the posterior 54 of the meniscus implant 12 within the inside segment 56 of the implant.

In a method of assembly, the keel 30, including the bone-anchoring portion 32 and intermediate portion 34 may be constructed using the various processes described herein. Once the keel 30 has been constructed, the keel may be placed within a mold cavity as discussed in U.S. patent application Ser. No. 11/448,954 and the other various references of which are incorporated by reference herein or known to those in the art. The mold cavity may include a forming area that enables a polymer material to be disposed therein. The polymer material is introduced into the forming area of the mold cavity and is allowed to cure into a desired shape so as to form the articular portion of the meniscal implant 12. During this process, the polymer material is also allowed to creep into the intermediate portion 34 of the keel 30. The intermediate portion 34 of the keel 30 preferably has a porosity that enables the polymer to adhere to the various metal lattice constructed within the intermediate portion. Once cured, the combination the polymer locked within the metal lattice of the intermediate portion 34 secures the articulation portion 20 to the bone-engaging portion 32 of the keel 30.

As described in the various references disclosed herein, the keel 30 may include areas with various gradient porosities as well as barrier layers and other features described in the references incorporated, which aid the keel in promoting bone ingrowth and the like including a desirable porosity and pore size.

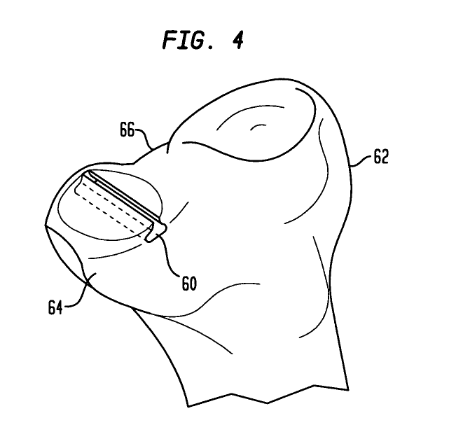

In a method of implantation, a small trough 60 of bone may be removed from a central section of the tibia adjacent to the tibial spine as shown in FIG. 4. The trough 60 preferably has a geometry that corresponds to the keel 30 or at least to the bone-anchoring portion 32 of the keel. The small trough 60 extends from the anterior 64 to the posterior 66 of the tibia. Thus, the trough 60 includes a lower portion 66 having a greater width than an upper portion 68 of the trough. This difference in width helps lock the meniscus implant within the tibial plateau as will be described shortly.

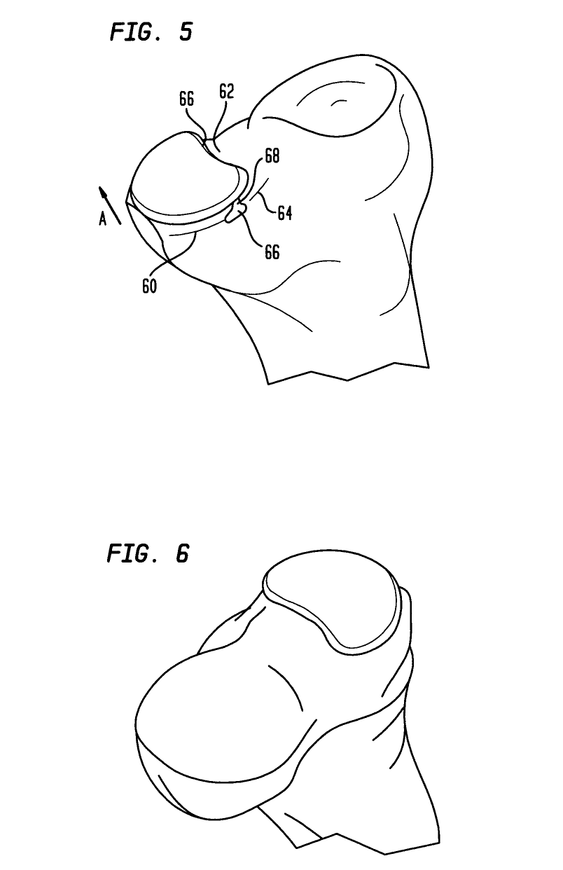

Once the small trough 60 has been created within the tibial plateau by methods known to those in the art, the meniscus implant 12 may be introduced to the tibial plateau. As shown in FIG. 5, the keel 30 may be received within the trough 60 by placing an edge of the keel adjacent a first edge of the small trough 60. The entire meniscus implant 12 may then be slid in a direction as denoted by arrow A such that the meniscus implant 12 enters the trough 60 in a anterior to posterior direction. Of course, the meniscus implant may also be slid in reverse if the surgeon is using a posterior to anterior approach. The meniscus implant is slid forward until the articulation portion 20 of the meniscus implant 12 is positioned in a desired location sitting atop the tibial plateau. The inferior portion of the implant also known as the keel 30 may be inserted through this trough, while the superior portion of the meniscal implant 12 also known as the articulation portion 20 sits a top the tibial plateau 62 of the tibia 64. Since the keel includes a wide portion 46 beneath the narrow portion 44, the keel is unable to be removed from its location by applying a force out of the tibia and upwards. The only way to remove the meniscus implant 12 is to slide the keel anteriorly or posteriorly until the entire keel is removed from the trough 60. The keel 30 is now firmly placed within the trough 60 and is surrounded by adjacent bone. And with the bone-securing portion 32 of the keel promoting bone ingrowth, the keel will become firmly locked within the bone over time. Of course, if the keel has certain geometric shapes such as straight walls, it may be lowered into the trough.

Since the keel includes a narrow portion and a wide portion, the keel is designed to fit into the small trough or key-way in a key like fashion so as to lock the meniscal implant 12 into the bone and prevent loosening. Therefore, the key way allows the meniscal device to be implanted into the proximal end of the tibia using a anterior to posterior approach. Once the keel 30 is positioned within a small trough or key-way, the tibial facing surface 16 of the implant is disposed adjacent and against the tibia. Thus the meniscus device sits atop the tibia condyle thereby providing a low profile articulation surface. Besides the various key and key-way method of locking the meniscus implant 12 within the bone, other short and/or long-term fixation methods may be used. Some of these methods include the use of bone screws, nails, staples, sutures, and/or hooks. In addition, porous metal or poly pegs, sheets, rectangles or other geometric shapes could be used by itself or in combination to fix the implant in place to allow for bone ingrowth. Further, bone cement could also be used to hold the implant to the bone; and ultrasonic waves may be used to cause protrusions on the backside of the implant to melt while being driven into the underlying bone. This would force the molten material to infuse and harden into the surrounding bone. The protrusions could be made of a homogenic material or other porous or solid metal reinforced plastic. Any additional property of the plastic protrusions could have the ability to be reabsorbed into the body and allow soft tissue or bone to grow into the implant for fixation. The metal reinforcements could be hollow, slotted or porous.

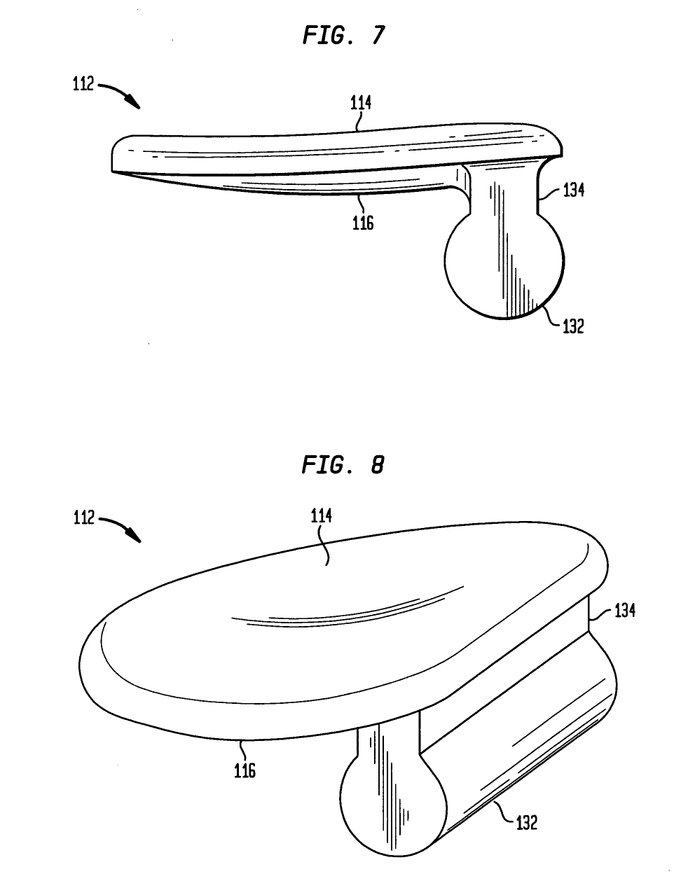

Another option may be to use small metal tubes or pegs that are slotted or porous and filled with a polymer material that could be melted or forced outside of the metal tubes after insertion into the bone. UV curing adhesives could be injected through openings in the implant after it is inserted into the bone and allowed to harden by inserting fiberoptic cables into the openings and activating the adhesive. A mesh could also be disposed on the tibia facing surface 16 to help secure the articulation portion 20 of the meniscus implant 12 to the tibial plateau. In yet another alternate embodiment as shown in FIGS. 7 and 8, a meniscal implant 112 is similarly structured as meniscal implant 12 and therefore includes a femoral facing surface 14 and an oppositely-facing tibial surface 16. These surfaces may be constructed similarly to the surfaces of meniscal implant 12 and form an articulation portion 120 of the implant 112.

The meniscal implant 112 also includes a keel 130, similar to keel 30 of meniscal implant 12. Although the keel 130 may have many different geometrical configurations as may keel 30, keel 130 is shown in FIGS. 7 and 8 has a circular bone-anchoring portion 132 and a rectangular intermediate portion 134. The bone anchoring portion 132 is substantially circular in side view but as shown in FIG. 8 is actually substantially cylindrical. The intermediate portion 134, although not shown in the figure, may extend into the cylindrical shape of the bone anchoring portion 32 such that the respective porosity of each portion is not limited to exactly being within the bone anchoring portion 132 or intermediate portion 134. Of course, when using a cylindrically shaped bone anchoring portion 132, any trough that is cut within the tibial plateau should have a similar geometric configuration or allow for the cylindrical bone anchoring portion 132 to be placed within the trough.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of performing surgery comprising:
Forming a groove in a tibial plateau;
Providing an implant having a bone-securing element and an articulation element; and
Placing said bone-securing element of said implant within said groove such that said articulation element is disposed adjacent the tibial plateau.

2. The method of claim 1, wherein said groove extends from the anterior of the tibia to the posterior.

3. The method of claim 1, further comprising removing at least some damaged cartilage from the tibial plateau.

4. The method of claim 1, wherein the groove includes a first width extending from a medial to lateral side and a second width extending in the same direction, wherein the first width is larger than the second width.

5. The method of claim 4, wherein the bone-securing element of the implant includes a corresponding geometric shape to the shape of the groove.

6. The method of claim 1, wherein the implant includes an intermediate portion that is attached to both the bone-securing element and the articulation element such that the intermediate portion connects the articulation element to the bone-securing element.

7. The method of claim 1, wherein the articulation element has a maximum height of 7 mm.

8. The method of claim 1, wherein the bone-securing element has a maximum height of 20 mm.

9. A meniscus implant comprising:
compressible bearing element having an articulation surface;
bone-securing element extending downwardly from the bearing element and configured to be engaged within a channel created within a tibial plateau.

10. The meniscus implant of claim 9, wherein at least a portion of the compressible bearing element is embedded within a portion of the bone-securing element.

11. The meniscus implant of claim 10, wherein the bone-securing element has a porosity that promotes bone ingrowth.

12. The meniscus implant of claim 11, wherein the bone-securing element includes a first side wall and a second side wall, each of said first and second side walls having a transition portion that transitions the first side and second side walls being separated by a first distance to the walls being separated by a second distance, the first distance being different than the second distance.

13. The meniscus implant of claim 12, wherein the implant includes an interior half positioned (new) to a mechanical axis of a tibia and a exterior half remote from the mechanical axis of the tibia, wherein the bone-securing element is positioned within the interior half of the implant.

14. The meniscus implant of claim 12, wherein the compressible bearing element is comprised of a polymer.

15. The meniscus implant of claim 9, wherein the compressible bearing element includes a concave articulation surface and a convex bearing surface.

16. The meniscus implant of claim 9, wherein the compressible bearing element has a maximum thickness of 10 mm.

17. The meniscus implant of claim 9, wherein the bone-securing element extends from near, or equal to the anterior of the implant to near, or equal to the posterior of the implant.
